(19) United States
(12) Patent Application Publication
(10) Pub. No.: US 2003/0220643 A1
(43) Pub. Date: Nov. 27, 2003

(54) Devices to prevent spinal extension

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/444,883

(22) Filed: May 23, 2003

Related U.S. Application Data
(60) Provisional application No. 60/383,041, filed on May 24, 2002.
(60) Provisional application No. 60/385,171, filed on May 31, 2002.

(51) Int. Cl. A61B 17/56

(52) U.S. Cl. 606/61

(57) ABSTRACT

This invention resides in apparatus for inhibiting full extension between upper and lower vertebral bodies, thereby preventing pain and other complications associated with spinal movement. In the preferred embodiment, the invention provides a generally transverse member extending between the spinous processes and lamina of the upper and lower vertebral bodies, thereby inhibiting full extension. Various embodiments of the invention may limit spinal flexion, rotation and/or lateral bending while preventing spinal extension. In the preferred embodiment, the transverse member is fixed between two opposing points on the lower vertebral body using pedicle screws, and a cushioning sleeve is used as a protective cover. The transverse member may be a rod or cable, and the apparatus may be used with a partial or full artificial disc replacement. To control spinal flexion, rotation and/or lateral bending one or more links may be fastened to an adjacent vertebral body, also preferably using a pedicle screw. Preferably a pair of opposing links are used between the upper and lower vertebral bodies for such purposes. Alternative embodiments use stretchable elements with or without a transverse member.

### REFERENCE TO RELATED APPLICATION

This application claims priority from U.S. Provisional Patent Application Serial No. 60/383,041, filed May 24, 2003, and No. 60/385,171, filed May 31, 2003. The entire content of each application is incorporated herein by reference.

### FIELD OF THE INVENTION

This invention relates generally to spine surgery and, in particular, to apparatus for preventing spinal extension.

### BACKGROUND OF THE INVENTION

Extension of the spine increases pressure on the facet joints, and this pressure may lead to low back pain in some patients, particularly those with arthritic facet joints.

A number of devices to prevent spinal extension have been described. One category of such devices allows spinal flexion but prevents spinal extension. Devices in this category can be divided into two broad subclasses. First, as described in U.S. Pat. No. 5,375,823, longitudinal shock absorbing devices can be connected to pedicle screws placed into adjacent levels of the spine. That is, paired longitudinal devices are placed into paired pedicles screws in the L3 and L4 vertebrae to prevent extension between L3 and L4.

Second, as described in U.S. Pat. Nos. 5,609,634 and 6,379,355, devices can be attached to the spinous processes between two vertebrae. For example, an elastomeric core may be strapped to the spinous processes of L3 and L4 to prevent spinal extension between L3 and L4.

### SUMMARY OF THE INVENTION

This invention resides in apparatus for inhibiting full extension between upper and lower vertebral bodies, thereby preventing pain and other complications associated with spinal movement. In the preferred embodiment, the invention provides a generally transverse member extending between the spinous processes and lamina of the upper and lower vertebral bodies, thereby inhibiting full extension. Various embodiments of the invention may limit spinal flexion, rotation and/or lateral bending while preventing spinal extension.

In the preferred embodiment, the transverse member is fixed between two opposing points on the lower vertebral body using pedicle screws, and a cushioning sleeve is used as a protective cover. The transverse member may be a rod or cable, and the apparatus may be used with a partial or full artificial disc replacement. To control spinal flexion, rotation and/or lateral bending one or more links may be fastened to an adjacent vertebral body, also preferably using a pedicle screw. Preferably a pair of opposing links are used between the upper and lower vertebral bodies for such purposes. Alternative embodiments use stretchable elements with or without a transverse member.

### BRIEF DESCRIPTION OF DRAWINGS

FIG. 1 is a view of the posterior portion of the spine utilizing the present invention;

FIG. 2 a view of the lateral portion of the spine and the present invention;

FIG. 3 is a view of the posterior portion of the spine and another embodiment of the present invention;

FIG. 4 is a lateral view of the spine and the embodiment of the device drawn in FIG. 3; and

FIG. 5 is a view of the lower end of the plate and the portion of the device that prevents extension;

FIG. 6A is a side view of an alternative embodiment of the invention including a spring connected to pedicle screws;

FIG. 6B is a view of the top of the device drawn in FIG. 6A;

FIG. 6C is a side view of the alternative embodiment of the invention drawn in FIG. 6A, including sleeves placed over the springs;

FIG. 6D is a view of the embodiment of the invention shown in FIG. 6C;

FIG. 7A is a sagittal cross section of a pedicle screw used in an alternative embodiment of the invention;

FIG. 7B is a view of the top of the screw drawn in FIG. 7A;

FIG. 7C is a side view of the embodiment of FIG. 7A, showing a telescoping rod connected to the screws by axles;

FIG. 8 is view of the side of an alternative embodiment of the invention including a rod or rods connected by mobile ball and socket joint coupled to pedicle screws;

FIG. 9A is a view of the side of the spine, an ADR, and an embodiment of the device similar to FIG. 6D;

FIG. 9B is a view of the side of a flexed spine, an ADR in flexion, and an embodiment of the device drawn in FIG. 9A;

FIG. 10 is a view of the side of another embodiment of the invention showing how an extension blocking sleeve can be placed over an elastic cord; and

FIG. 11 is a partial sagittal cross section of another embodiment of the invention including an elastic member connected to pedicle screws that allow for spinal flexion.

### DETAILED DESCRIPTION OF THE INVENTION

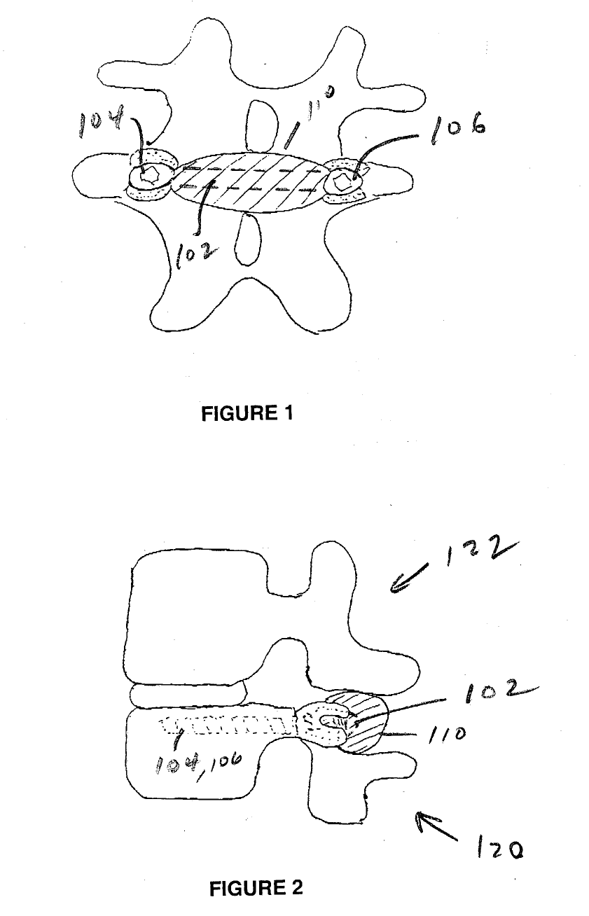

This invention minimizes or eliminates spinal extension through the use of a generally transverse member, preferably anchored at two points on the same vertebra to prevent adjacent vertebrae from coming closer together. The preferred embodiment, shown in FIGS. 1 and 2, uses a sleeved rod 102 or cable placed transversely between pedicle screws 104, 106. The sleeve 110 impinges upon the spinous processes and lamina of the vertebra 120 holding the pedicle screws and the spinous processes and lamina of the vertebra 122 directly above the vertebra with the pedicle screws 104, 106. As such, the device prevents spinal extension at the level it is inserted. For example, the device would be placed between pedicle screws at L4 to prevent extension of the spine between L3 and L4.

The sleeve could be made of any compressible or resilient material that has cushion-like properties and sufficient tensile strength. For example, natural and synthetic rubbers, elastomers, encased gels and foams, and fiber-reinforced polymers may be acceptable.

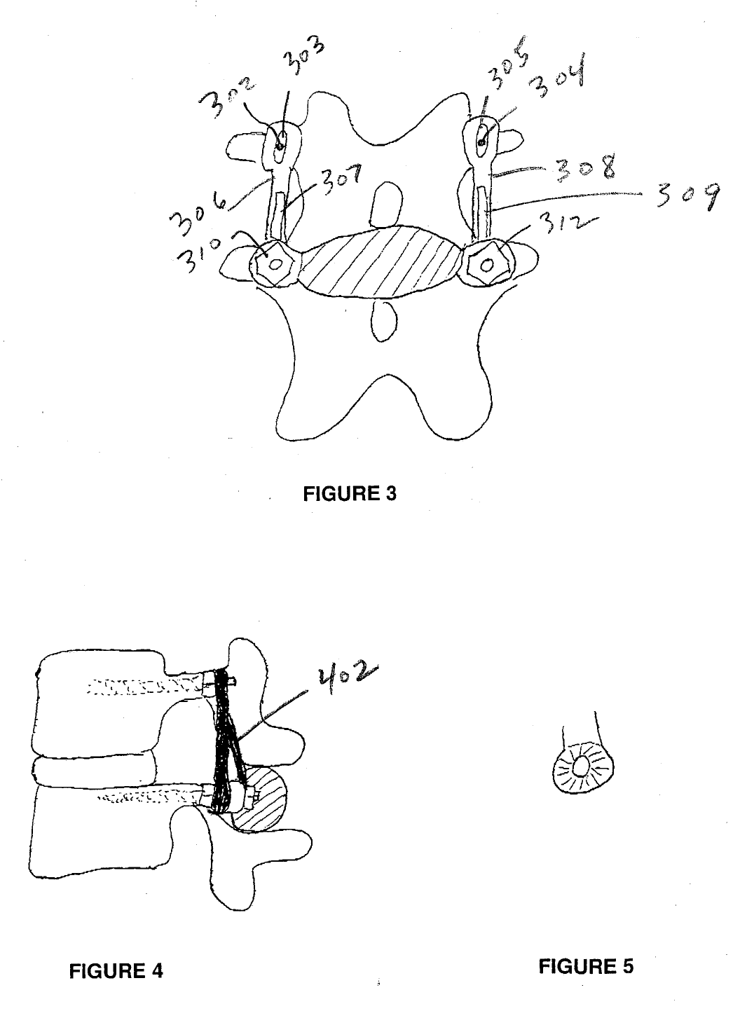

FIG. 3 is a view of the posterior portion of the spine and another embodiment of the invention, wherein pedicle screws 302, 304 are placed into the upper vertebra. Metal plates extend from lower pedicle screws 310, 312 to the upper pedicle screws. The upper portions of the plates preferably include a slot 303, 305 to guide the movement of the upper vertebra relative to the screws 302, 304. In this way, lateral bending and rotation between the vertebrae may be limited. Thus, the extension stop prohibits extension and the plate eliminates other movements that could cause pain from the facet joints. Flexion need not be limited by either portion of the device.

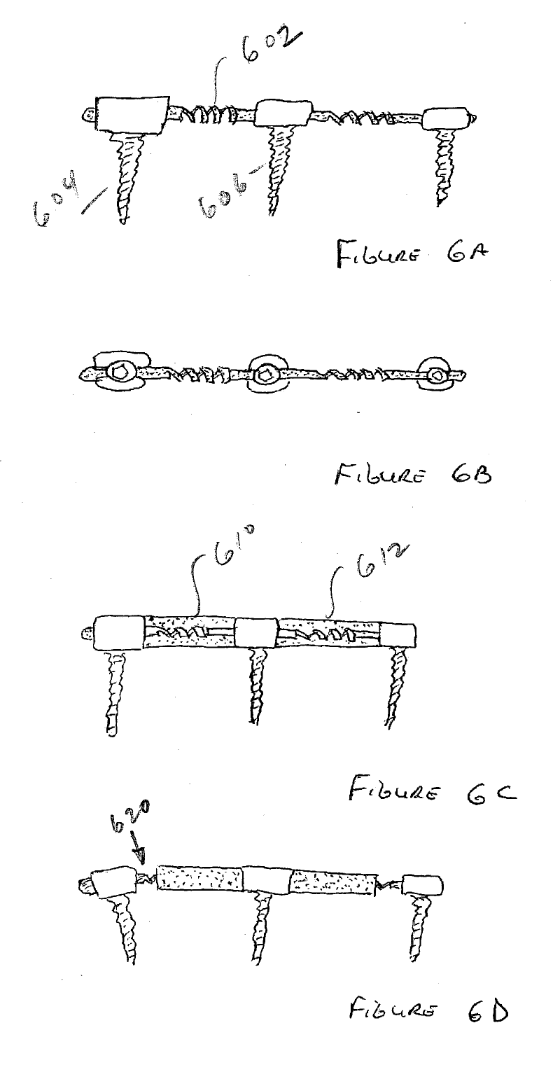

FIG. 4 is a lateral view of the spine and the embodiment of the device drawn in FIG. 3, showing how each metal plate may include a brace 402 to help the plate control the movement of the upper vertebra. FIG. 5 is a view of the lower end of the plate and the portion of the device that prevents extension. Both components could have serrated teeth that interdigitate with each other and the lower pedicle screw to help prevent rotation of the plate.

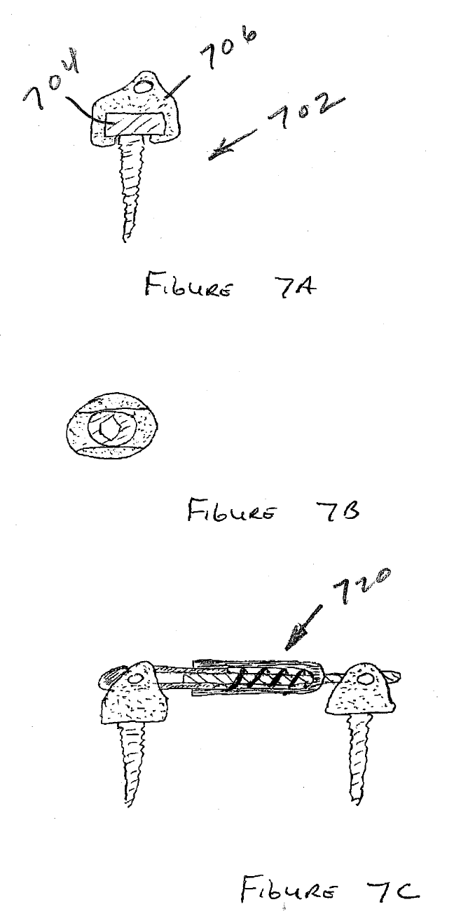

FIG. 6A is a view of the side of an alternative embodiment of the invention including a spring 602 connected to pedicle screws 604, 606. The spring allows spinal flexion and a limited degree of lateral bending and axial rotation while preventing spinal extension without the need for a transverse member. FIG. 6B is a view of the top of the device drawn in FIG. 6A. FIG. 6C is a side view of the alternative embodiment of the invention drawn in FIG. 6A, including sleeves 610, 612 placed over the springs. Impingement between the sleeves and the pedicle screws assists the spring in preventing spinal extension. The sleeves may be constructed of any appropriate material, including those listed herein above. FIG. 6D is a view of the embodiment of the invention drawn in FIG. 6C. The drawing illustrates a gap 620 that forms between the pedicle screws and the sleeves with spinal flexion.

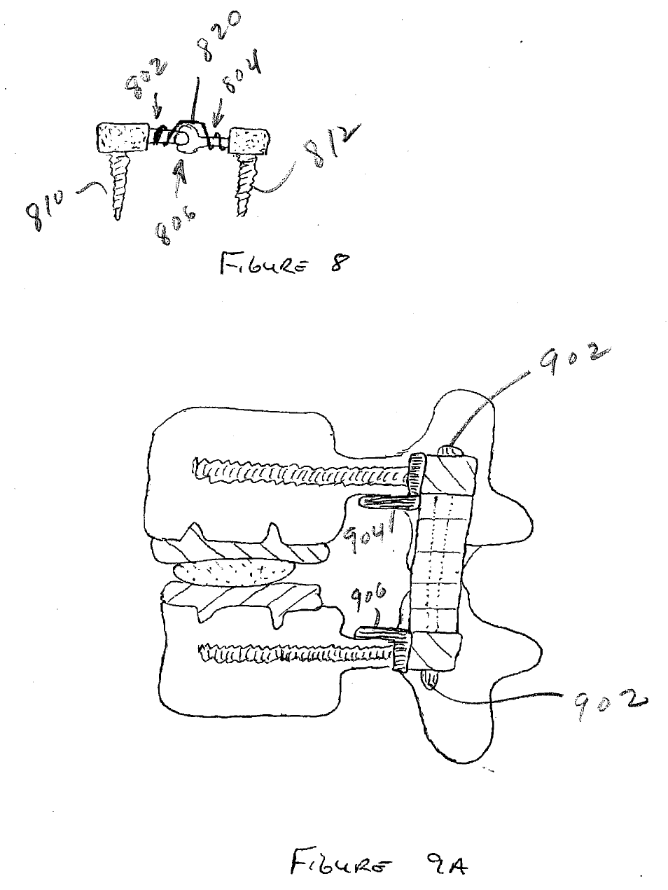

FIG. 7A is a sagittal cross section of a pedicle screw 702 used in an alternative embodiment of the invention. A screw with a cylinder-shaped head 704 sits within a connector with a receiving cylinder 706, allowing the screw to axially rotate within the connector. FIG. 7B is a view of the top of the screw drawn in FIG. 7A. FIG. 7C is a side view of the embodiment of FIG. 7A, showing a telescoping rod 720 connected to the screws by axles. The spring over the rod allows flexion, but inhibits spinal extension. The sleeve over the spring helps prevent tissue ingrowth.

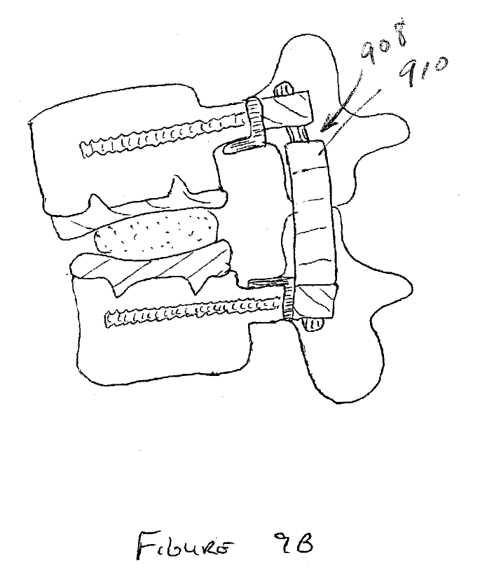

FIG. 8 is view of the side of an alternative embodiment of the invention including a rod or rods connected by mobile ball and socket joint 806 coupled to pedicle screws 810, 812. The ball and socket joint 806 are surrounded by a spring 820 to facilitate spinal flexion, limited lateral bending, and limited spinal rotation. The cooperation between the rods and the springs inhibits spinal extension.

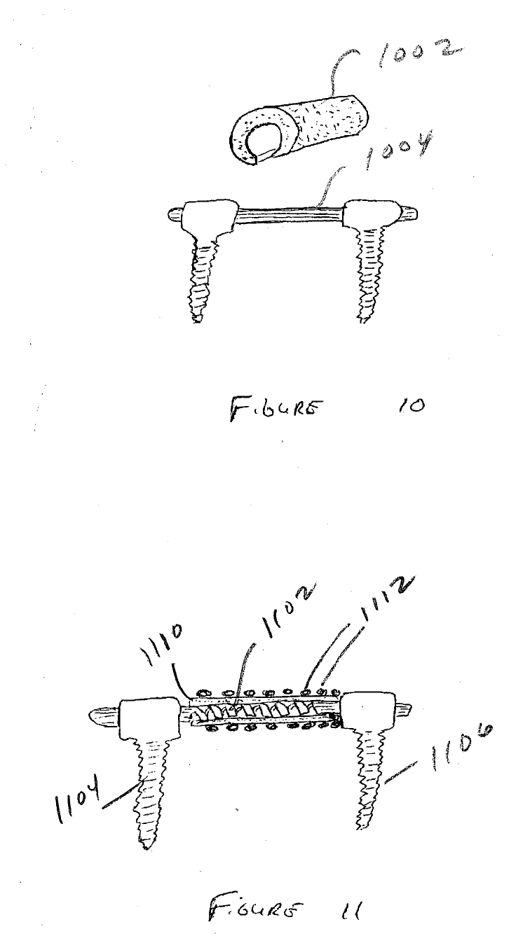

FIG. 9A is a view of the side of the spine, an ADR, and an embodiment of the device similar to FIG. 6D. The spring in the device of FIG. 6D has been replaced with an elastic cord 902. L-shaped braces 904, 906 can be seen extending from the pedicle screws to the sides of the pedicle screws. The L-shaped braces place loads on the sides of the pedicles with spinal extension. FIG. 9B is a view of the side of a flexed spine, an ADR in flexion, and an embodiment of the device drawn in FIG. 9A. Note gap 908 between the sleeve 910 and the superior pedicle screw with spinal flexion.

FIG. 10 is a view of the side of another embodiment of the invention showing how an extension blocking sleeve 1002 can be placed over the elastic cord 1004 after the elastic cord is positioned in the pedicle screws 1006, 1008. The extension blocking sleeves plastically deform as they are snapped over the cord. The sleeves could be made of metal or polymers such as polyethylene or other materials, including those listed herein above, which are capable of plastic deformation. The sleeves may also be made of shape-memory alloys such as Nitinol, allowing the sleeves to change shape once they are positioned over the elastic cord. The Nitinol sleeves would contract around the elastic cord once the sleeves are placed in the body.

FIG. 11 is a partial sagittal cross section of another embodiment of the invention including an elastic member 1102 connected to pedicle screws 1104, 1106 that allows spinal flexion. The first elastic member may be covered by a sleeve 1110 which, in turn may be surrounded by a second, more rigid member 1112 used to prevent spinal extension. Springs may be used as the first and second members 1102, 1112. The sleeve 1110 prevents one spring from catching on the second spring. The second spring, or outer spring, is drawn in cross section.

The invention is not limited to use in conjunction with natural vertebral discs, and may be used with partial or full artificial disc replacements (ADRs). The motion preserving embodiments for posterior insertion that allow at least partial spinal flexion while inhibiting at least a certain degree of spinal extension may be particularly suited for use with an ADR. Patients may experience low back pain following ADR insertion. The pain may be related to arthritis of the facet joints. The device and method according to this invention attempt to preserve ADR motion while decreasing the forces across the facet joints, thereby mitigating such problems.

I claim:

1. Apparatus for preventing full extension between upper and lower vertebral bodies, each having spinous processes and lamina, the apparatus comprising:
a generally transverse member extending between the spinous processes and lamina of the upper and lower vertebral bodies, thereby inhibiting full extension.

2. The apparatus of claim 1, wherein the device is fixed at two opposing points on the lower vertebral body.

3. The apparatus of claim 2, wherein the device is fixed using pedicle screws.

4. The apparatus of claim 1, further including a cushioning sleeve covering the transverse member.

5. The apparatus of claim 1, wherein the transverse member is a rod or cable.

6. The apparatus of claim 1, further including a partial or full artificial disc replacement.

7. The apparatus of claim 1, further including a link to another vertebral body to inhibit spinal flexion, rotation, or lateral bending.

8. The apparatus of claim 7, wherein the link is fastened to the upper vertebral body using a pedicle screw.

9. The apparatus of claim 7, including a pair of opposing links extending between the upper and lower vertebral bodies.

10. Apparatus for preventing full extension between upper and lower vertebral bodies, comprising:
a stretchable element connected between the upper and lower vertebral bodies in a compressed state, thereby allowing at least a limited degree of spinal flexion, lateral bending and axial rotation while inhibiting spinal extension.

11. The apparatus of claim 10, wherein the stretchable element is a spring.

12. The apparatus of claim 10, wherein the stretchable element is attached to the respective vertebral bodies using pedicle screws.

13. The apparatus of claim 10, further including a sleeve covering the stretchable element.

14. The apparatus of claim 13, further including a second stretchable element outside the sleeve.

15. The apparatus of claim 10, further including a partial or full artificial disc replacement.

16. The apparatus of claim 10, including a pair of opposing stretchable elements extending between the upper and lower vertebral bodies.
